(19) United States
(12) Patent Application Publication
(10) Pub. No.: US 2026/0266669 A1
(43) Pub. Date: Sep. 10, 2026

(54) Thermocouple Device

(71) Applicants: Measurement Specialties (Chengdu) Ltd., Chengdu (CN); TE Connectivity Solutions GmbH, Schaffhausen (CH)

(72) Inventors: Zhijiao CHE, Chengdu (CN); Brian Clark, Dayton, MN (US); Robert Karaba, St. Francis, MN (US)

(73) Assignees: Measurement Specialties (Chengdu) Ltd., Chengdu (CN); TE Connectivity Solutions GmbH, Schaffhausen (CH)

(21) Appl. No.: 19/556,055

(22) Filed: Mar. 4, 2026

(30) Foreign Application Priority Data
Mar. 4, 2025 (CN) 202510249662.0

(51) Int. Cl. G01K 13/25; A61B 18/00; C09D 127/04; G01K 7/04; H10N 10/01

(52) U.S. Cl. CPC G01K 13/25; A61B 18/00; G01K 7/04; H10N 10/01; A61B 2018/00351; A61B 2018/00577; A61B 2018/00821; C09D 127/04

(57) ABSTRACT

A thermocouple device includes a thermocouple and a conformal coating that covers a surface of the thermocouple. The thermocouple has a positive electrode, a negative electrode, and a thermal junction connecting the positive electrode and the negative electrode. The positive electrode and the negative electrode are respectively formed at a pair of ends of a pair of parallel thermocouple wires. The conformal coating is biocompatible and completely covers the positive electrode, the negative electrode, and the thermal junction.

### CROSS-REFERENCE TO RELATED APPLICATION

This application claims the benefit of the filing date under 35 U.S.C. § 119(a)-(d) of Chinese Patent Application No. 202510249662.0, filed on Mar. 4, 2025.

### FIELD OF THE INVENTION

The invention relates to the field of temperature sensors, and in particular to a thermocouple device with a conformal coating.

### BACKGROUND OF THE INVENTION

The technical means of sealing and insulating a surface of a micro thermocouple usually adopt the following forms: the first is a structure of heat shrink wrapping of the sleeve, which is usually made of polyethylene terephthalate (PET) material, and the second is a structure of sleeve plus glue filling, which is usually made of polyimide (PI) or PET material, and the glue filling material is usually epoxy resin.

These thermocouples can only meet certain insulation requirements, for example of current ablation catheter products, with a withstand voltage of 50VDC and an insulation strength greater than 10 MΩ, but cannot meet insulation requirements of an insulation strength greater than 5 MΩ at 500VAC, and cannot meet new minimum withstand voltage requirements of 1500VDC, and preferably 2500VAC for the new generation of ablation catheter products. In addition, the sleeve structure can only be applied to thermocouple wires with a wire diameter greater than 44 AWG. The size of the sleeve cannot be made smaller to cover a thermocouple wire with a smaller wire diameter. The thickness dimension tolerance of the sleeve cannot be controlled within a smaller tolerance range, which has an adverse effect on its set operation on the thermocouple. In addition, since the wire and sleeve of the thermocouple wire are very soft and very small in size, the length of the sleeve is limited, and the covering length is also limited, which cannot meet the customer's requirements for a longer covering length. Further, the structure of sleeve heat shrink wrapping or sleeve plus glue filling is unstable in molding, and the insulation layer formed on the surface of the thermocouple has poor uniformity and consistency, which is prone to insulation withstand voltage failure. The head material of the thermocouple also easily accumulates and exceeds the specified size, resulting in the client being unable to install it. Excessive heat shrinkage of the sleeve is likely to cause the sleeve to be trapped or ruptured, resulting in insulation strength that cannot meet the requirements. During the curing process of the sleeve plus glue filling, bubbles are likely to be generated, or contaminants are brought in, resulting in poor insulation performance of the product, or the appearance does not meet customer requirements.

There is a multi-point polymer encapsulated thermocouple in the prior art, in which a heat shrinkable polymer material is used to form an electrically insulating seal on the micro-thermocouple. To form this seal, a tube made of the polymer material is slid onto the hot junction, and then the tube made of the polymer material is heated to a melting point and a seal is formed on the hot junction and the insulating sheath of the wire. The polymer material is melted and a dome shape is formed on the top of the micro-thermocouple.

There is also a thermocouple wire for measuring the internal temperature of a battery pack and an armored thermocouple for measuring a battery pack, for which the thermocouple wire has a withstand voltage of up to 3,000 VDC. The surfaces of these thermocouples are coated with polyparaxylene high temperature (HT) coating, but the coating material polyparaxylene HT contains fluorine, and such materials cannot meet the European Union's restrictions on per-and polyfluoroalkyl substances (PFAS).

### SUMMARY OF THE INVENTION

A thermocouple device includes a thermocouple and a conformal coating that covers a surface of the thermocouple. The thermocouple has a positive electrode, a negative electrode, and a thermal junction connecting the positive electrode and the negative electrode. The positive electrode and the negative electrode are respectively formed at a pair of ends of a pair of parallel thermocouple wires. The conformal coating is biocompatible and completely covers the positive electrode, the negative electrode, and the thermal junction.

### BRIEF DESCRIPTION OF THE DRAWINGS

Features of the present invention will become more apparent by describing in detail exemplary embodiments thereof with reference to the accompanying drawings, in which:

FIG. 1 is a schematic perspective view of a thermocouple device according to an exemplary embodiment; and

FIG. 2 is a schematic perspective view of a thermocouple device including a plurality of thermocouples and a plurality of pairs of thermocouple wires according to an exemplary embodiment.

### DETAILED DESCRIPTION

The technical solution of the present invention is further specifically described below by examples and in conjunction with the accompanying drawings. In the specification, the same or similar reference numerals indicate the same or similar components. The following description of the embodiments of the present invention with reference to the accompanying drawings is intended to explain the overall inventive concept of the present invention and should not be construed as a limitation of the present invention.

In addition, in the detailed description below, for ease of explanation, many specific details are set forth to provide a comprehensive understanding of the disclosed embodiments. However, it is apparent that one or more embodiments may be implemented without these specific details. In other cases, known structures and devices are embodied in a schematic manner to simplify the accompanying drawings.

According to an overall technical concept of the present invention, a thermocouple device is provided, characterized in that it includes: a thermocouple suitable for sensing the temperature of muscle tissue; and a conformal coating covering the surface of the thermocouple, wherein the conformal coating satisfies biocompatibility.

The advantage of the thermocouple device according to the present invention is that the coating of the thermocouple device is thinner than that of the sleeve and the glue potting, and the coating material is a material that meets biocompatibility, which can ensure medical-grade hygiene requirements. The coating of the present invention is applied by a chemical deposition process, and the covering size is accurate. The size of the thermocouple device of the present invention can be made very small while still having high pressure resistance and insulation. The conformal coating of the present invention has good uniformity and consistency, so that the performance of the thermocouple device is stable and controllable. The thermocouple that can be coated with the conformal coating of the present invention has a wide applicable wire diameter range, and can be suitable for coating double-row single thermocouple wires and multi-row thermocouple wires.

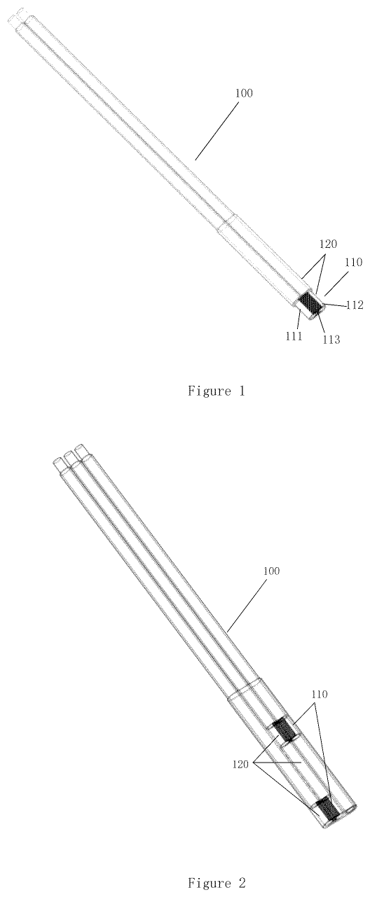

A thermocouple device 100 according to an embodiment, as shown in FIG. 1, includes a thermocouple 110 suitable for sensing the temperature of muscle tissue, and a conformal coating 120 covering the surface of the thermocouple 110, wherein the conformal coating 120 is biocompatible or meets biocompatibility.

The thermocouple device 100, in an embodiment, is disposed in an ablation catheter and inserted into human tissue together with the ablation catheter. The thermocouple device 100 can sense the temperature of the ablation catheter head and thus the muscle tissue around the ablation catheter.

The coating 120 of the thermocouple device 100 according to the present invention is thinner than a sleeve and glue potting, and the coating material is a material that meets biocompatibility, which can ensure medical-grade hygiene requirements. In an embodiment, the conformal coating 120 is made of parylene, which is a medical grade material and meets biocompatibility. In an embodiment, the conformal coating 120 is made of parylene C or parylene D or other polymer materials that meet biocompatibility requirements. The conformal coating 120 of the present invention adopts the above-mentioned material and has good uniformity and consistency, so that the performance of the thermocouple device 100 is stable and controllable.

A thickness of the conformal coating 120 is between 12 μm and 25 μm. For example, the dielectric strength of the thermocouple device 100 with a film thickness of 12 μm is 1500V AC, and the dielectric strength of the thermocouple device 100 with a film thickness of 25 μm is 2500V AC. Therefore, the thermocouple 110 of the present invention must meet the application threshold of 1500VDC. The above-mentioned coating thickness range can prevent the thermocouple device 100 of the present invention from being broken down during use.

The conformal coating 120 may be formed on the thermocouple 110 by a chemical deposition process. According to the present invention, the polyparaxylene or parylene material is coated on the surface of the thermocouple 110 by a chemical vapor deposition process (CVD), which can obtain a thinner, more consistent and more uniform insulation layer than conventional sleeve and glue potting technology.

As shown in FIG. 1, the thermocouple 110 according to the present invention includes a positive electrode 111, a negative electrode 112, and a hot junction 113 connecting the top ends of the positive electrode 111 and the negative electrode 112, that is, a temperature measurement node, wherein the positive electrode 111 and the negative electrode 112 are respectively formed at the ends of a pair of parallel thermocouple wires, and the conformal coating 120 completely covers the positive electrode 111, the negative electrode 112 and the hot junction 113. A thermal contact is located at a distal end of the thermocouple wires and is connected to a measurement system.

As shown, the conformal coating 120 also covers the thermocouple wire formed thereon with the thermocouple 110, wherein the conformal coating 120 covers a length of 0.25 inches to 1.25 inches along the length direction of the thermocouple 110. In this way, the thermocouple device 100 can be protected over a longer area.

In an embodiment, a diameter of the thermocouple 110 wire is between AWG36 and AWG50.

In an embodiment, the thermocouple device 100 is suitable for being inserted into an ablation catheter (not shown) to sense the temperature change of the head of the ablation catheter and the muscle tissue around the head of the ablation catheter. The thermocouple device 100 of the present invention can be installed in an ablation catheter to monitor temperature changes during atrial fibrillation and other arrhythmia surgeries. Since pulsed electric field ablation technology is applied in ablation catheters, the catheters will generate a pulsed high voltage of at least 1500VDC, or even up to 2400VDC during ablation surgery. The coating 120 thickness of the thermocouple device 100 of the present invention can meet the withstand voltage value between 1500VAC-2500VAC, and can also meet the special regulations of China. The insulation requirement of 500VDC greater than 5 MΩ in the current standard of Chinese radiofrequency ablation catheters, the actual insulation strength test result of the thermocouple device 100 of the present invention is greater than 100 MΩ at 500VDC.

The thermocouple device 100 according to the present invention is not limited to a single thermocouple 110 but, as shown in FIG. 2, may also include a plurality of thermocouples 110 and a plurality of pairs of thermocouple wires, wherein the thermocouple device 100 is a plurality of pairs of thermocouples 110 in a wire arrangement. The conformal coating 120 covers the multiple thermocouples 110 of the wire-arranged multiple-pair thermocouple 110 and the multiple pairs of thermocouple wires on which the thermocouples 110 are formed.

Those skilled in the art can understand that the embodiments described above are exemplary and other embodiments not specifically described herein are within the scope of the present invention. The structures described in various embodiments can be freely combined without causing conflicts in structure or principle.

According to the thermocouple device of the present invention, the parylene coating is used to replace the conventional sleeve/glue filling technical solution, so that the dielectric strength can be as high as 2500V, the formed coating is uniform and has good consistency, and the thickness of the coating can be easily controlled within a very small tolerance range. In addition, the insulating coating of the present invention is coated by a chemical deposition process, can be formed at room temperature, and the covering size can also be very precise. The conformal coating of the present invention can be applied to thermocouple wires with a wire diameter less than 44 AWG, and the coating of the present invention meets biocompatibility, complies with Restriction of Hazardous Substances (RoHS) standards and Registration, Evaluation, Authorisation, and Restriction of Chemicals (REACH) regulations, is free of PFAS materials, and is more environmentally friendly. The present invention meets the newly added voltage resistance requirement of a new generation of ablation catheter products, which is a minimum of 1500VDC, preferably 2500VAC.

In the aforementioned exemplary embodiments of the present invention, the coating of the thermocouple device according to the present invention is thinner than the sleeve and the glue potting, and the coating material adopts a material that meets the biocompatibility, which can ensure the medical grade hygiene requirements. The size of the thermocouple device of the present invention can be made very small while still having high pressure resistance and insulation. The thermocouple that can be coated with the conformal coating of the present invention has a wide applicable wire diameter range, and can be suitable for coating double-row single thermocouple wires and multi-row thermocouple wires.

Although the present invention has been described in conjunction with the accompanying drawings, the embodiments disclosed in the accompanying drawings are intended to exemplify implementations of the present invention and should not be construed as limiting the present invention.

Although some embodiments of the present general inventive concept have been shown and described, it will be appreciated by those skilled in the art that changes may be made to these embodiments without departing from the principles and spirit of the present general inventive concept, the scope of which is defined by the claims and their equivalents.

It should be noted that the word “comprising” does not exclude other elements or steps, and the word “a” or “an” does not exclude a plurality. In addition, any element reference signs in the claims shall not be construed as limiting the scope of the present invention.

1. A thermocouple device, comprising:
a thermocouple including a positive electrode, a negative electrode, and a thermal junction connecting the positive electrode and the negative electrode, the positive electrode and the negative electrode are respectively formed at a pair of ends of a pair of parallel thermocouple wires; and
a conformal coating that covers a surface of the thermocouple, the conformal coating is biocompatible and completely covers the positive electrode, the negative electrode, and the thermal junction.

2. The thermocouple device of claim 1, wherein the conformal coating is a parylene material.

3. The thermocouple device of claim 2, wherein the conformal coating is a parylene C or a parylene D material.

4. The thermocouple device of claim 1, wherein the conformal coating has a thickness between 12 μm and 25 μm.

5. The thermocouple device of claim 1, wherein the conformal coating is deposited on the thermocouple by chemical deposition.

6. The thermocouple device of claim 1, wherein the conformal coating covers a length of 0.25 inches to 1.25 inches of the thermocouple along a length direction of the thermocouple.

7. The thermocouple device of claim 1, wherein a wire diameter of each of the pair of parallel thermocouple wires is between AWG36 and AWG50.

8. The thermocouple device of claim 1, wherein the thermocouple is one of a plurality of thermocouples.

9. The thermocouple device of claim 8, wherein the plurality of thermocouples include a plurality of pairs of thermocouple wires and are arranged into the thermocouple device.

10. The thermocouple device of claim 8, wherein the conformal coating covers the plurality of thermocouples.

11. The thermocouple device of claim 1, wherein the thermocouple is adapted to sense a temperature of a muscle tissue.

12. The thermocouple device of claim 11, wherein the thermocouple device is insertable into an ablation catheter and senses temperatures changes of a head of the ablation catheter and the muscle tissue around the head.

13. A method, comprising:
providing a thermocouple device including a thermocouple and a conformal coating covering a surface of the thermocouple, the thermocouple includes a positive electrode, a negative electrode, and a thermal junction connecting the positive electrode and the negative electrode, the positive electrode and the negative electrode are respectively formed at a pair of ends of a pair of parallel thermocouple wires, the conformal coating is biocompatible and completely covers the positive electrode, the negative electrode, and the thermal junction; and
sensing a temperature of a muscle tissue using the thermocouple device.

14. The method of claim 13, further comprising forming the conformal coating on the thermocouple by a chemical deposition process.

15. The method of claim 13, further comprising inserting the thermocouple device into an ablation catheter.

16. The method of claim 15, wherein the sensing step includes sensing a temperature change of a head of the ablation catheter and the muscle tissue around the head.
